(19) United States
(12) Patent Application Publication
(10) Pub. No.: US 2015/0224177 A1
(43) Pub. Date: Aug. 13, 2015

(54) METHODS FOR THERAPEUTIC OR PROPHYLACTIC TREATMENT OF MELIOIDOSIS AND/OR ASSOCIATED DISEASES

(71) Applicants: STRAGEN PHARMA SA, Geneva (CH); ALAXIA SAS, Lyon (FR)

(72) Inventors: Philippe BORDEAU, St Pierre De Chandieu (FR); Pascale GAILLARD, Lyon (FR)

(21) Appl. No.: 14/617,309

(22) Filed: Feb. 9, 2015

Related U.S. Application Data
(60) Provisional application No. 61/937,765, filed on Feb. 10, 2014.

(51) Int. Cl. A61K 38/40; A61K 33/18; A61K 45/06; A61K 31/275

(52) U.S. Cl. CPC A61K 38/40; A61K 31/275; A61K 33/18; A61K 45/06

(57) ABSTRACT

The present invention generally relates to a method for therapeutic or prophylactic treatment of melioidosis and/or associated diseases in a subject in need thereof, comprising administering to said subject an effective amount of a pharmaceutical composition comprising either one ion selected from the group of the hypothiocyanites (OSCN⁻) and/or hypohalites and/or lactoferrin or a combination thereof.

The present invention also generally relates to methods of treating or preventing various bacterial infections selected from the group consisting of Burkholderia pseudomallei, Burkholderia mallei, bacillus anthracis, Yersinia pestis, and francisella tularensis infections.

### CROSS REFERENCE TO RELATED APPLICATIONS

This application claims priority to U.S. provisional patent application No. 61/937,765, entitled: “Methods for Therapeutic or Prophylactic treatment of Melioidosis and/or associated diseases,” filed Feb. 10, 2014, which is hereby incorporated by reference into this disclosure.

### FIELD OF THE INVENTION

The present invention generally relates to a method for therapeutic or prophylactic treatment of melioidosis and/or associated diseases in a subject in need thereof, comprising administering to said subject an effective amount of a pharmaceutical composition comprising either one ion selected from the group of the hypothiocyanites (OSCN⁻) and/or hypohalites or lactoferrin or a combination′ thereof.

The present invention also generally relates to methods of treating or preventing various bacterial infections selected from the group consisting of Burkholderia pseudomallei, Burkholderia mallei, bacillus anthracis, Yersinia pestis, and Francisella tularensis infections.

### BACKGROUND ART

Melioidosis is a highly fatal infectious bacterial disease, primarily occurring in rodents in India and Southeast Asia that is characterized in humans by systemic caseous nodules. In particular melioidosis is an infectious disease of humans and animals caused by a gram-negative bacillus found in soil and water. It has both acute and chronic forms. Melioidosis is endemic (occurring naturally and consistently) in Southeast Asia, Australia, and parts of Africa. It was rare in industrialized countries prior to recent immigrations.

Melioidosis, also called Whitmore's Disease, is an infectious disease caused by a bacterium called Burkholderia pseudomallei (previously known as Pseudomonas pseudomallei). The bacteria are found in contaminated water and soil and spread to humans and animals through direct contact with the contaminated source.

Melioidosis is presently a public health concern because it is most common in AIDS patients and intravenous drug users. Burkholderia pseudomallei, is a bacillus that can cause disease in sheep, goats, pigs, horses, and other animals, as well as in humans. The bacterium that causes the disease is found in the soil, rice paddies, and stagnant waters of the area. Infection most commonly occurs during the rainy season. The organism enters the body through skin abrasions, burns, or wounds infected by contaminated soil; inhalation of dust; or by eating food contaminated with B. pseudomallei. Person-to-person transmission is unusual. Drug addicts acquire the disease from shared needles. The incubation period is two to three days.

Melioidosis most commonly involves the lungs where the infection can form a cavity of pus (abscess). The bacteria can also spread from the skin through the bloodstream the brain, eyes, heart, liver, kidneys, and joints. The common symptoms of melioidosis are not specific. They include headaches, fever, chills, cough, chest pain, and loss of appetite. Melioidosis can also cause encephalitis (brain inflammation) with seizures (convulsions).

Chronic melioidosis is characterized by osteomyelitis (inflammation of the bone) and pus-filled abscesses in the skin, lungs, or other organs.

Acute melioidosis takes one of three forms: a localized skin infection that may spread to nearby lymph nodes; an infection of the lungs associated with high fever, headache, chest pain, and coughing; and septicemia (blood poisoning) characterized by disorientation, difficulty breathing, severe headache, and an eruption of pimples on the head or trunk. The third form is most common among drug addicts and may be rapidly fatal. Melioidosis is usually suspected based on the patient's history, especially travel, occupational exposure to infected animals, or a history of intravenous drug. Diagnosis must then be confirmed through laboratory tests. B. pseudomallei can be cultured from samples of the patient's sputum, blood, or tissue fluid from abscesses. Blood tests, including complement fixation (CF) tests and hemagglutination tests, also help to confirm the diagnosis. In acute infections, chest x rays and liver function tests are usually abnormal.

Patients with mild or moderate infections are given a course of trimethoprim-sulfamethoxazole (TMP/SMX) and ceftazidime by mouth. Patients with acute melioidosis are given a lengthy course of ceftazidime followed by TMP/SMX. In patients with acute septicemia, a combination of antibiotics is administered intravenously, usually tetracycline, chloramphenicol, and TMP/SMX. The mortality rate in acute cases of pulmonary melioidosis is about 10%; the mortality rate for the septicemic form is significantly higher (slightly above 50%).

The interest of hypothiocyanite ions is no longer to be shown for either the agri-food industry or for the pharmaceutical industry. The hypothiocyanite and/or hypohalite ion is in particular generated in vivo by the lactoperoxidase system, according to the equation below:

$${H}_{2} {O}_{2} + {SCN}^{-} ( and \text{/} or {I}^{-} ) {\to }^{ lactoperoxidase } {OSCN}^{-} ( and \text{/} or {OI}^{-} )$$

The pharmacological properties of the hypothiocyanite ion, particularly its biocidal properties, are well known, but owing to the instability of this chemical species, the half-life thereof is about 24 hours, it has not been possible to develop any formulation enabling local pulmonary treatment under satisfactory conditions.

For example, from WO2007134180 a therapeutic composition acting through the action of the hypothiocyanite ion, comprising an enzyme system, for example an oxidoreductase which produces hydrogen peroxide by reduction of a specific substrate, the specific substrate, for example glucose, the SCN⁻ ion and lactoperoxidase is known. The difficulty in formulating such therapeutic compositions is understood, as are the side effects that may be produced, for example here in the respiratory system by the in vivo production of hydrogen peroxide, which has an inflammatory and genotoxic effect and cannot be administered in long-term treatments.

In US2002/172645 the thiocyanate ion is administered alone to feed the endogenous lactoperoxidase system and form hypothiocyanite ions in vivo, or as in WO2007134180 in combination with the lactoperoxidase system.

The properties of lactoferrin are in any case well known, in particular its action on biofilms and its anti-inflammatory action. From WO 2008/003688 the demonstration of a synergy between the hypothiocyanite ion and the lactoferrin is known. International application WO 2010/086530 relates to the use of a synergistic combination of at least one selected from the group of hypothiocyanites and/or hypohalite and lactoferrin for the preparation of a pharmaceutical composition for the treatment of cystic fibrosis ion.

In one embodiment the lactoferrin is a lactoferrin of greater than 95% purity, substantially free of lipopolysaccharide, endotoxin and angiogenin and a saturation level of greater than 15% iron.

However at the present time no satisfactory formulation has been developed that enables a local treatment and particularly the destruction of bacteria which develop in patients suffering from melioidosis and other related diseases, and in particular on Burkholderia pseudomallei, which is highly pathogenic and particularly difficult to eradicate especially due to antibiotic resistant strains.

### BRIEF DESCRIPTION OF THE INVENTION

This section provides a general summary of the disclosure, and is not a comprehensive disclosure of its full scope or all of its features.

The present invention generally relates to the use of a pharmaceutical composition comprising either one ion selected from the group of the hypothiocyanites (OSCN⁻) and/or hypohalites and/or lactoferrin or a combination thereof for treating or preventing melioidosis and other associated diseases.

In one embodiment, the invention relates to the use of a synergistic combination of at least one ion selected from the group of the hypothiocyanites (OSCN⁻) and/or hypohalites and lactoferrin for preparing a pharmaceutical composition for the treatment or prevention of melioidosis infections caused by at least one bacterium selected from the group consisting of Burkholderia pseudomallei.

The present invention also generally relates to methods of treating or preventing various associated diseases comprising bacterial infections selected from the group consisting of Burkholderia mallei, bacillus anthracis, Yersinia pestis, and francisella tularensis infections.

Consequently in another embodiment, the present invention further concerns the use of a pharmaceutical composition comprising either one ion selected from the group of the hypothiocyanites (OSCN⁻) and/or hypohalites or lactoferrin or a combination thereof for treating or preventing infections selected from the group consisting of Burkholderia mallei, bacillus anthracis, Yersinia pestis, and francisella tularensis infections.

Thus in one preferred embodiment, the invention relates to the use of a synergistic combination of at least one ion selected from the group of the hypothiocyanites (OSCN⁻) and/or hypohalites and lactoferrin for preparing a pharmaceutical composition for the treatment or prevention of infections caused by at least one bacterium selected from the group consisting of Burkholderia pseudomallei. Burkholderia mallei, bacillus anthracis, Yersinia pestis, and francisella tularensis infections.

### BRIEF DESCRIPTION OF THE FIGURES

FIG. 1 illustrates the evolution of Strain 1026b during time 1^{st} repetition (Kill curve 1).

The kill curve assay was done in biological triplicate for B. pseudomallei strain (1026b & MSHR 305) on three different days, and each dilution of the samples was plated in technical triplicate at each time point.

The initial inoculum (T₀ₕ) was between 1.33×10⁶ and 2.43×10⁶ CFU/mL for 1026b. As expected, the bacterial counts in all of the positive control samples continued to increase throughout the time course, while the negative controls had no growth at any of the time.

OSCN curve shows immediate decrease of microbial load after 2 hours. OSCN+bLF curve shows low level of load and eradication at T₂₄ₕ (OSCN, bLF or OSCN+bLF are added at T₀ only). bLF impact can be seen at T₆ₕ (standalone) and with OSCN+bLF at T₂₄ₕ

FIG. 2 shows the evolution of Strain 1026b during time 2^{nd} repetition (Kill curve 2).

The kill curve assay was done in biological triplicate for B. pseudomallei strain (1026b & MSHR 305) on three different days, and each dilution of the samples was plated in technical triplicate at each time point.

The initial inoculum (T₀ₕ) was between 1.33×10⁶ and 2.43×10⁶ CFU/mL for 1026b. As expected, the bacterial counts in all of the positive control samples continued to increase throughout the time course, while the negative controls had no growth at any of the time.

OSCN curve shows immediate decrease of microbial load after 2 hours. OSCN+bLF curve shows low level of load at T₂₄ₕ (OSCN, bLF or OSCN+bLF are added at T₀ only).

bLF impact can be seen at T₆ₕ (standalone) and with OSCN+bLF at T₂₄ₕ

FIG. 3 shows the evolution of Strain 1026b during time 3^{rd} repetition (Kill curve 3).

The kill curve assay was done in biological triplicate for B. pseudomallei strain (1026b & MSHR 305) on three different days, and each dilution of the samples was plated in technical triplicate at each time point.

The initial inoculum (T₀ₕ) was between 1.33×10⁶ and 2.43×10⁶ CFU/mL for 1026b. As expected, the bacterial counts in all of the positive control samples continued to increase throughout the time course, while the negative controls had no growth at any of the time.

OSCN curve shows immediate decrease of microbial load after 2 hours. OSCN+bLF curve shows eradication at T₂₄ₕ (OSCN, bLF or OSCN+bLF are added at T₀ only).

bLF impact can be seen at T₄ₕ (standalone) and with OSCN+bLF at T₂₄ₕ

FIG. 4 illustrates the evolution of Strain MSHR 305 during time 1^{st} repetition (Kill curve 1).

The kill curve assay was done in biological triplicate for B. pseudomallei strain (1026b & MSHR 305) on three different days, and each dilution of the samples was plated in technical triplicate at each time point.

The initial inoculum (T₀ₕ) was between 8.33×10⁵ and 3.67×10⁶ CFU/mL for MSHR. As expected, the bacterial counts in all of the positive control samples continued to increase throughout the time course, while the negative controls had no growth at any of the time.

OSCN curve shows immediate decrease of microbial load after 2 hours. OSCN+bLF curve shows low level of load and eradication at T₂₄ₕ (OSCN, bLF or OSCN+bLF are added at T₀ only).

bLF impact can be seen at T₆ₕ (standalone) and with OSCN+bLF at T₂₄ₕ

FIG. 5 shows the evolution of Strain MSHR 305 during time 2^{nd} repetition (Kill curve 2).

The kill curve assay was done in biological triplicate for B. pseudomallei strain (1026b & MSHR 305) on three different days, and each dilution of the samples was plated in technical triplicate at each time point.

The initial inoculum (T₀ₕ) was between 8.33×10⁵ and 3.67×10⁶ CFU/mL for MSHR. As expected, the bacterial counts in all of the positive control samples continued to increase throughout the time course, while the negative controls had no growth at any of the time.

OSCN curve shows immediate decrease of microbial load after 2 hours. OSCN+bLF curve shows eradication at T₂₄ₕ (OSCN, bLF or OSCN+bLF are added at T₀ only).

bLF impact can be seen at T₄ₕ (standalone) and with OSCN+bLF at T₂₄ₕ

FIG. 6 shows the evolution of Strain MSHR 305 during time 3^{rd} repetition (Kill curve 3).

The kill curve assay was done in biological triplicate for B. pseudomallei strain (1026b & MSHR 305) on three different days, and each dilution of the samples was plated in technical triplicate at each time point.

The initial inoculum (T₀ₕ) was between 8.33×10⁵ and 3.67×10⁶ CFU/mL for MSHR. As expected, the bacterial counts in all of the positive control samples continued to increase throughout the time course, while the negative controls had no growth at any of the time.

OSCN curve shows immediate decrease of microbial load after 2 hours. OSCN+bLF curve shows eradication at T₂₄ₕ (OSCN, bLF or OSCN+bLF are added at T₀ only).

bLF impact can be seen at T₄ₕ (standalone) and with OSCN+bLF at T₂₄ₕ

### DETAILED DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control.

Unless defined otherwise, all technical and scientific terms used herein have the same meaning as is commonly understood by one of skill in art to which the subject matter herein belongs.

As used herein, the following definitions are supplied in order to facilitate the understanding of the present invention.

“A” or “an” means “at least one” or “one or more.”

The term “comprise” is generally used in the sense of include, that is to say permitting the presence of one or more features or components.

As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “an effective amount” refers to an amount necessary to obtain a physiological effect. The physiological effect may be achieved by one application dose or by repeated applications. The dosage administered may, of course, vary depending upon known factors, such as the physiological characteristics of the particular composition; the age, health and weight of the subject; the nature and extent of the symptoms; the kind of concurrent treatment; the frequency of treatment; and the effect desired and can be adjusted by a person skilled in the art.

As used herein, the terms “prevention” and “preventing,” when referring to a disorder or symptom, refers to a reduction in the risk or likelihood that a mammalian subject will develop said disorder, symptom, condition, or indicator after treatment according to the invention, or a reduction in the risk or likelihood that a mammalian subject will exhibit a recurrence of said disorder, symptom, condition, or indicator once a subject has been treated according to the invention and cured or restored to a normal state.

As used herein, the terms “treatment” or “treating,” when referring to, melioidosis and other associated diseases refers to inhibiting or reducing the progression, nature, or severity of the subject condition or delaying the onset of the condition.

The present invention generally relates to the use of a pharmaceutical composition comprising either one ion selected from the group of the hypothiocyanites (OSCN⁻) and/or hypohalites or lactoferrin or a combination thereof for treating or preventing melioidosis and other associated diseases comprising bacterial infections selected from the group consisting of Burkholderia mallei, bacillus anthracis, Yersinia pestis, and francisella tularensis infections.

Usually, hypothiocyanite can be either in liquid or solid form.

In a preferred embodiment, the invention relates to the use of a synergistic combination of at least one ion selected from the group of the hypothiocyanites (OSCN⁻) and/or hypohalites and lactoferrin for preparing a pharmaceutical composition for the treatment or prevention of infections caused by at least one bacterium selected from the group consisting of Burkholderia pseudomallei. Burkholderia mallei, bacillus anthracis, Yersinia pestis, and francisella tularensis infections.

Burkholderia mallei is a gram-negative bipolar aerobic bacterium, a Burkholderia-genus human and animal pathogen causing Glanders; the Latin name of this disease (malleus) gave name to the causative agent species. It is closely related to B. pseudomallei, and by multilocus sequence typing, it is a subspecies of B. pseudomallei. B. mallei evolved from B. pseudomallei by selective reduction and deletions from the B. pseudomallei genome. Unlike closely related Burkholderia pseudomallei and other genus members, the bacterium is non-motile; its shape is something in between a rod and a coccus measuring some 1.5-3 μm in length and 0.5-1 μm in diameter with rounded ends.

B. mallei is responsible for causing Glanders disease, which historically affected animals, such as horses, mules, and donkeys the most, and rarely affected humans. Horses are considered the natural host for B. mallei infection and are highly susceptible to it. B. mallei infects and gains access to the cell of its host through lysis of the entry vacuole. B. mallei has bacterial protein dependent actin-based motility once inside the cell. It is also able to initiate host cell fusion that results in multi-nucleated giant cells (MNGCs). The consequence of MNGCs has yet to be determined, but it may allow the bacteria to spread to different cells, evade responses by the infected host's immune system, or allow the bacteria to remain in the host longer. B. mallei is able to survive inside host cells through its capabilities in disrupting the bacteria killing functions of the cell. It leaves the vacuoles early, which allows for efficient replication of the bacteria inside the cell.

Leaving the cell early also keeps the bacteria from being destroyed by lysosomal defensins and other pathogen killing agents. MNGCs may help protect the bacteria from immune responses. B. mallei's ability to live within the host cell makes developing a vaccine against it difficult and complex. The vaccine would need to create a cell-mediated immune response as well as a humoral response to the bacteria in order to be effective in protecting against B. mallei.

Horses who are chronically infected with B. mallei and have Glanders disease as a result, typically experience mucus containing nasal discharge, lung lesions, and nodules around the liver or spleen. Acute infection in horses results in a high fever, loss of fat or muscle, erosion of the surface of the nasal septum, hemorrhaging or mucus discharge. The bacterium mostly affects the lungs and airways. Human infection with B. mallei is rare, although it occasionally occurs among lab workers dealing with the bacteria or those who are frequently near infected animals. The bacteria usually infect a person through their eyes, nose, mouth, or cuts in the skin. Once a person is infected with the bacteria, they develop a fever and rigors. Eventually they will get pneumonia, pustules, and abscesses, which will prove fatal within a week to ten days if left untreated by antibiotics. The way someone is infected by the bacteria also affects the type of symptoms that will result. If the bacteria enter through the skin, a local skin infection can result, while inhaling B. mallei can cause septicemic or pulmonary infections of muscles, the liver, or spleen. B. mallei infection has a fatality rate of 95% if left untreated, and a 50% fatality rate in individuals treated with antibiotics.

B. mallei as well as B. pseudomallei have a history of being on a list of potential biological agents. The Centers for Disease Control and Prevention (CDC) classifies B. mallei as a Category B critical biological agent. It is so highly infective and a potential biological weapon, little research has been conducted on this bacterium.

Bacillus anthracis is the etiologic agent of anthrax, a common disease of livestock and, occasionally, of humans and the only obligate pathogen within the genus Bacillus. B. anthracis is a Gram-positive, endospore-forming, rod-shaped bacterium, with a width of 1-1.2 μm and a length of 3-5 μm. It can be grown in an ordinary nutrient medium under aerobic or anaerobic conditions. It is one of few bacteria known to synthesize a protein capsule (poly-D-gamma-glutamic acid). Like Bordetella pertussis, it forms a calmodulin-dependent adenylate cyclase exotoxin known as (oedema factor), along with lethal factor. It bears close genotypical and phenotypical resemblance to Bacillus cereus and Bacillus thuringiensis. All three species share cellular dimensions and morphology. All form oval spores located centrally in an unswollen sporangium. B. anthracis spores in particular are highly resilient, surviving extremes of temperature, low-nutrient environments, and harsh chemical treatment over decades or centuries. The spore is a dehydrated cell with thick walls and additional layers that form inside the cell membrane. It can remain inactive for many years, but if it comes into a favorable environment, it begins to grow again. It is sometimes called an endospore, because it initially develops inside the rod-shaped form. Features such as the location within the rod, the size and shape of the endospore, and whether or not it causes the wall of the rod to bulge out are characteristic of particular species of Bacillus.

Depending upon the species, the endospores are round, oval, or occasionally cylindrical. They are highly refractile and contain dipicolinic acid. Electron micrograph sections show that they have a thin outer spore coat, a thick spore cortex, and an inner spore membrane surrounding the spore contents. The spores resist heat, drying, and many disinfectants (including 95% ethanol). B. anthracis possesses a capsule that is antiphagocytic and is essential for full virulence. The organism also produces three plasmid-coded exotoxins: edema factor, a calmodulin-dependent adenylate cyclase, causes elevation of intracellular cAMP, and is responsible for the severe edema usually seen in B. anthracis infections; lethal toxin is responsible for tissue necrosis; protective antigen (so named because of its use in producing protective anthrax vaccines) mediates cell entry of edema factor and lethal toxin.

Three forms of human anthrax disease are recognized based on their portal of entry.
Cutaneous, the most common form (95%), causes a localized, inflammatory, black, necrotic lesion (eschar).
Pulmonary, the highly fatal form, is characterized by sudden, massive chest oedema followed by cardiovascular shock.
Gastrointestinal, a rare but also fatal (causes death to 25%) type, results from ingestion of spores.

Yersinia pestis (formerly Pasteurella pestis) is a Gram-negative rod-shaped coccobacillus, a facultative anaerobic bacterium that can infect humans and other animals.

Human Y. pestis infection takes three main forms: pneumonic, septicemic, and bubonic plagues. All three forms are widely believed to have been responsible for a number of high-mortality epidemics throughout human history, including the Justinianic plague of the sixth century and the Black Death that accounted for the death of at least one-third of the European population between 1347 and 1353. It has now been shown conclusively that these plagues originated in rodent populations in China. More recently, Y. pestis has gained attention as a possible biological agent and the CDC has classified it as a category “A pathogen”. Every year, thousands of cases of plague are still reported to the World Health Organization, although, with proper treatment, the prognosis for victims is now much better. A five- to six-fold increase in cases occurred in Asia during the time of the Vietnam war possibly due to the disruption of ecosystems and closer proximity between people and animals. Plague also has a detrimental effect on non-human mammals. In the United States of America, animals such as the black-tailed prairie dog and the endangered black-footed ferret are under, threat from the disease.

There are three forms of the plague that commonly occur worldwide: bubonic, septicemic, and pneumonic. Bubonic plague is easily diagnosed by the presence of extremely swollen and tender lymph glands called “buboes” that can grow to the size of an egg, and typically arise in the groin, neck and armpits. Disease becomes evident 2-6 days after infection, and carries symptoms such as high fevers, chills, headache, and extreme exhaustion. One nasty side effect is the development of gangrene in the extremities, lending it the name “Black Death”. Bacteremia and death from Gram-negative induced shock occurs in 40-60% of untreated cases, while only 1-10% of treated cases are lethal. Septicemic plague often develops secondarily to bubonic plague, and is a result of direct invasion of the bloodstream without involvement of the lymph nodes. Due to the lack of buboes, symptoms generally resemble the flu and make diagnosis difficult. In severe cases, seizure and shock can take place. Death rates for this form are 40% for treated cases and 100% for untreated cases. The most serious form of infection is the pneumonic plague, which is 100% lethal if not treated within the first 24 hours. This mode of infection is the result of inhaled droplets of infectious material that proceed to directly colonize the lung tissue. Symptoms, on top of those found in the other two forms, include a severe cough, bloody sputum, chest pains, confusion, cyanosis, shock and eventual death.

Francisella tularensis is a pathogenic species of Gram-negative bacteria and the causative agent of tularemia, the pneumonic form of which is often lethal without treatment. It is a fastidious, facultative intracellular bacterium which requires cysteine for growth. Due to its low infectious dose, ease of spread by aerosol and high virulence, F. tularensis is classified as a Class A Select Agent by the U.S. government, along with other potential lethal agents such as Yersinia pestis, Smallpox and Ebola.

F. tularensis has been reported in birds, reptiles, fish, invertebrates and mammals including humans. Despite this, no case of tularemia has been shown to be initiated by human-to-human transmission. Rather, tularemia is caused by contact with infected animals or vectors such as ticks, mosquitos, and deer flies. Reservoir hosts of importance can include lagomorphs, rodents, galliform birds and deer.

Infection with F. tularensis can occur via several routes. The most common occurs via skin contact, yielding an ulceroglandular form of the disease. Inhalation of bacteria particularly biovar tularensis, leads to the potentially lethal pneumonic tularemia. While the pulmonary and ulceroglandular forms of tularemia are more common, other routes of inoculation have been described and include oropharyngeal infection due to consumption of contaminated food and conjunctival infection due to inoculation at the eye.

F. tularensis is capable of surviving outside of a mammalian host for weeks at a time and has been found in water, grassland, and haystacks. Aerosols containing the bacteria may be generated by disturbing carcasses due to brush cutting or lawn mowing; as a result, tularemia has been referred to as lawnmower disease. Recent epidemiological studies have shown a positive correlation between occupations involving the above activities and infection with F. tularensis.

When the U.S. biological warfare program ended in 1969 F. tularensis was one of seven standardized biological weapons it had developed.

The present invention generally relates to a method for therapeutic or prophylactic treatment of melioidosis and/or associated diseases in a subject in need thereof, comprising administering to said subject an effective amount of a pharmaceutical composition comprising either:

a) one ion selected from the group of the hypothiocyanites (OSCN⁻) and/or hypohalites or

b) lactoferrin,

or a combination thereof.

In accordance with the invention, melioidosis “associated or related diseases” are defined as bacterial infections caused by bacterial selected from the group consisting of Burkholderia pseudomallei, Burkholderia mallei, bacillus anthracis, Yersinia pestis, and francisella tularensis infections.

Preferably, the ion is the hypothiocyanite ion (OSCN⁻). In particular, hypothiocyanite can be either in liquid or solid form.

In another embodiment, the hypohalite ions are selected from the group consisting of the hypoiodite, hypochlorite and hypobromite ions.

Preferably, the ion is the hypoiodite ion (OI⁻).

In another embodiment, the lactoferrin is a bovine lactoferrin and preferably having a purity higher than 97%, essentially free from endotoxin, lipopolysaccharide and angiogenin and with an iron saturation level lower than 15% and preferably lower than 10%.

The pharmaceutical composition of the invention may further comprise an antibiotic or a therapeutic agent. Preferably the therapeutic agent is a drug or an antibody.

In a particular embodiment, the pharmaceutical composition according to the invention is combined/associated or administered together with antibiotics for antibiotic potentiation and faster infection clearance. Usually the combined antibiotics are the one commonly used for the prevention or treatment of bacterial infections selected from the group consisting of Burkholderia pseudomallei, Burkholderia mallei, bacillus anthracis, Yersinia pestis, and francisella tularensis infections. Surprisingly it has been shown that the composition according to the invention presents a potentiating effect on the antibiotics' activity, resulting either in lowering the effecting amount of antibiotics that is usually required for the treatment, or in facilitating the destruction of antibiotic resistant strains, or in reducing the time of treatment and consequently the costs.

Preferably the antibiotics may belong to the following classes selected among Aminoglycosides, Ansamycins, β-Lactam, Carbacephem, Carbapenems, Cephalosporins, Glycopeptides, Lincosamides, Lipopeptide, Macrolides, Monobactams, Nitrofurans, Oxazolidonones, Penicillins, Polypeptides having an antibiotic activity, Quinolones, Rifamycins, Streptogramins, Sulfonamides, Sulfonamides, Tetracycline, Tuberactinomycins.

In particular, antibiotics according to the present invention may be selected from the group consisting of piperacillin, ceftazidime, temocillin, carbapenem, imipenem, meropenem, rifampicin, tobramycin, ciprofloxacin, monosulfactam, amoxicillin, carbenicillin, Doxycycline, penicillin, Trimethoprim-sulfamethoxazole, monobactam, Streptomycin, Fosfomycin, Ethionamide, Isoniazid, Pyrazinamide, Rifampicin, Rifabutin, Rifapentine, Arsphenamine, Chloramphenicol, Fusidic acid, Metronidazole, Mupirocin, Platensimycin, Quinupristin/Dalfopristin, Thiamphenicol, Tigecycline, Tinidazole, Trimethoprim or their combinations.

In a further embodiment, the invention relates to the use of a synergistic combination of at least one ion selected from the group of the hypothiocyanites (OSCN⁻) and/or hypohalites and/or lactoferrin for preparing a pharmaceutical composition for the treatment or prevention of melioidosis infections caused by at least one bacterium selected from the group consisting of Burkholderia pseudomallei.

The present invention also generally relates to methods of treating or preventing various diseases comprising bacterial infections selected from the group consisting of Burkholderia mallei, bacillus anthracis, Yersinia pestis, and francisella tularensis infections.

Consequently in another embodiment, the present invention further concerns the use of a pharmaceutical composition comprising either one ion selected from the group of the hypothiocyanites (OSCN⁻) and/or hypohalites or lactoferrin or a combination thereof for treating or preventing infections selected from the group consisting of Burkholderia mallei, bacillus anthracis, Yersinia pestis, and francisella tularensis infections.

Thus in one preferred embodiment, the invention relates to the use of a synergistic combination of at least one ion selected from the group of the hypothiocyanites (OSCN⁻) and/or hypohalites and lactoferrin for preparing a pharmaceutical composition for the treatment or prevention of infections caused by at least one bacterium selected from the group consisting of Burkholderia pseudomallei. Burkholderia mallei, bacillus anthracis, Yersinia pestis, and francisella tularensis infections.

The invention also relates to a method for therapeutic or prophylactic treatment of melioidosis and other associated diseases selected from the group consisting of Burkholderia mallei, bacillus anthracis, Yersinia pestis, and francisella tularensis infections, characterized in that for local treatment of the pulmonary epithelium it comprises the administration of a therapeutically active quantity of at least one ion selected from the group of the hypothiocyanites and/or hypohalites and/or lactoferrin or a combination thereof.

In certain cases, the bacteria develop on the epithelium of the lungs and the treatment must be local, hence the administration will be carried out orally and/or nasally and/or by any other artificial route enabling access to the lung, for example tracheotomy.

In one embodiment, the ion is the hypothiocyanite ion (OSCN⁻).

In another embodiment, the ion is the hypoiodite ion (OI⁻).

In a further embodiment, the lactoferrin is a lactoferrin having a purity higher than 97%, essentially free from endotoxin, lipopolysaccharide and angiogenin and with an iron saturation level lower than 15% and preferably lower than 10%.

Without being bound by theory, it is believed that the compositions according to the invention act by the following mechanisms:
the lactoferrin/OSCN⁻ combination destroys the bacteria and/or prevents their growth, and thus has a bacteriostatic and bactericidal effect.
In case where bacteria form biofilms, the lactoferrin destroys these biofilms.
The lactoferrin/OSCN⁻ combination administered in association with antibiotics acts as an enhancer of their efficacy (antibiotic booster).

The combination of the lactoferrin with the hypothiocyanite ion on the one hand makes it possible to reduce the concentration of hypothiocyanite in order to achieve the same anti-microbial effectiveness, and on the other hand to add the anti-inflammatory aspect to the antimicrobial aspect or to have a faster effect.

In a preferred embodiment of the invention, the method further comprises the administration of an antibiotic.

Usually the combined/associated antibiotics are the one commonly used for the prevention or treatment of bacterial infections selected from the group consisting of Burkholderia pseudomallei, Burkholderia mallei, bacillus anthracis, Yersinia pestis, and francisella tularensis infections.

Preferably the antibiotics may belong to the following classes selected among Aminoglycosides, Ansamycins, β-Lactam, Carbacephem, Carbapenems, Cephalosporins, Glycopeptides, Lincosamides, Lipopeptide, Macrolides, Monobactams, Nitrofurans, Oxazolidonones, Penicillins, Polypeptides having an antibiotic activity, Quinolones, Rifamycins, Steptogramins, Sulfonamides, Sulfonamides, Tetracyclines, Tuberactinomycins.

In particular, antibiotics according to the present invention may be selected from the group consisting of piperacillin, ceftazidime, temocillin, carbapenem, imipenem, meropenem, rifampicin, tobramycin, ciprofloxacin, monosulfactam, amoxicillin, carbenicillin, Doxycycline, penicillin, Trimethoprim-sulfamethoxazole, monobactam, Streptomycin, Fosfomycin, Ethionamide, Isoniazid, Pyrazinamide, Rifampicin, Rifabutin, Rifapentine, Arsphenamine, Chloramphenicol, Fusidic acid, Metronidazole, Mupirocin, Platensimycin, Quinupristin/Dalfopristin, Thiamphenicol, Tigecycline, Tinidazole, Trimethoprim or their combinations.

The invention also relates to a pharmaceutical composition intended for the treatment of the acute phases of melioidosis, characterized in that it comprises a volume of a solution of OSCN ion ranging from 2 to 10 mL at concentrations ranging from 800 to 5000 μM and Lactoferrin at doses ranging from 10 to 200 mg and more preferably 10 ml of a solution of OSCN⁻ ion at a concentration of 4000 μM and 100 mg of Lactoferrin.

The invention also relates to a pharmaceutical composition intended for prophylactic treatment of melioidosis, characterized in that it comprises for example 800 μM of the OSCN⁻ ion and 50 mg of Lactoferrin.

In a particular embodiment, the pharmaceutical composition for daily local administration to the pulmonary epithelium in the treatment of melioidosis and/or associated diseases may comprise for example about 4000 μM of OSCN⁻ ion, about 18 mM of SCN⁻ ion and about 100 mg of lactoferrin, wherein the composition comprises less than 1 ppm of each of glucose oxidase, lactoperoxidase, and hydrogen peroxide; and wherein the composition is suitable for administration via inhalation from a sprayer, nebulizer or aerosolizer at a volume of 1 ml to 100 ml of the final solution per inhalation or per broncho alveolar lavage so as to reach target sites within the lungs.

Preferably the associated diseases are caused by at least one bacterium selected from the group consisting of Burkholderia pseudomallei, Burkholderia mallei, bacillus anthracis, Yersinia pestis, and francisella tularensis.

In a preferred embodiment of the invention, the pharmaceutical composition further comprises the combination with antibiotics as described above.

The pharmaceutical composition of the invention may be associated with a pharmaceutically acceptable carrier. For instance the pharmaceutical composition are suitable for a topical, oral, sublingual, parenteral, intranasal, intravenous, intramuscular, subcutaneous, transcutaneous or intraocular administration and the like.

Preferably, the pharmaceutical composition contains vehicles which are pharmaceutically acceptable for a formulation capable of being injected.

The suitable pharmaceutical composition may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses of the composition used for the administration can be adapted as a function of various parameters, and in particular as a function of the mode of administration used, of the relevant pathology, or alternatively of the desired duration of treatment.

“Pharmaceutically” or “pharmaceutically acceptable” refer to molecular entities and compositions that do not produce an adverse, allergic or other untoward reaction when administered to an animal, including a human, as appropriate.

As used herein, a “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Dispersions can also be prepared in glycerol, liquid polyethylene glycols, and mixtures thereof and in oils. Under ordinary conditions of storage and use, these preparations contain a preservative to prevent the growth of microorganisms.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with a variety of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In addition to the formulations for parenteral administration, such as intravenous or intramuscular injection, other pharmaceutically acceptable forms include, e.g. tablets or other solids for oral administration; liposomal formulations; time release capsules; and any other form currently used, including creams.

Other routes of administration are contemplated, including nasal solutions or sprays, aerosols or inhalants, or vaginal or rectal suppositories and pessaries or cream, and long-acting delivery polymers.

The invention also relates to pharmaceutical compositions above defined further comprising a second pharmaceutical agent that acts synergistically with the composition of the invention.

The invention also relates to a method of administration of the composition according to the invention according to a dosage schedule characterized in that it comprises the twice daily administration of 5 ml of a formulation comprising 4000 μM of the OSCN⁻ ion and 100 mg of Lactoferrin during acute phase and possibly 800 μM of the OSCN and 50 mg of lactoferrin in prophylactic approach.

The invention also relates to a method of administration of the composition according to the invention following a dosage schedule characterized in that it comprises the twice daily administration of 5 ml of a formulation comprising 4000 μM of the OSCN⁻ ion and 100 mg of Lactoferrin as complemented therapy for example for a period of four weeks of treatment.

The invention also relates to a method of administration of the composition according to the invention following a dosage schedule characterized in that it comprises the twice daily administration of 10 ml of a formulation comprising 4000 μM of the OSCN⁻ ion and 100 mg of Lactoferrin as complemented therapy for example for a period of one to 2 weeks of treatment in acute infection.

In particular, the invention concerns the use of a combination comprising (a) at least one ion selected from the group of the hypothiocyanites and/or hypohalites and/or (b) lactoferrin for preparing a pharmaceutical composition suitable for local administration to the pulmonary epithelium in the long-term treatment of melioidosis and/or associated diseases, wherein the lactoferrin has a purity higher than 97%, essentially free from endotoxin, lipopolysaccharide and angiogenin and with an iron saturation level lower than 15% and preferably lower than 10%, wherein the pharmaceutical composition comprises 1 mg of lactoferrin for every 40 μM of ion (a), and wherein the composition comprises less than 1 ppm of each of glucose oxidase, lactoperoxidase, and hydrogen peroxide.

Preferably the associated diseases are caused by at least one bacterium selected from the group consisting of Burkholderia pseudomallei. Burkholderia mallei, bacillus anthracis, Yersinia pestis, and francisella tularensis.

Preferably, the ion is the hypothiocyanite ion (OSCN⁻).

In another embodiment of the invention, the ion is the hypoiodite ion (OI⁻).

Another object of the invention is to provide a method for therapeutic or prophylactic treatment of melioidosis and/or associated diseases, characterized in that for local treatment of the pulmonary epithelium it comprises the administration of a therapeutically active quantity of a synergistic combination of at least one ion selected from the group of the hypothiocyanites and/or hypohalites and of lactoferrin.

Preferably the ion is the hypothiocyanite ion and the lactoferrin is a lactoferrin having a purity higher than 97%, essentially free from endotoxin, lipopolysaccharide and angiogenin and with an iron saturation level lower than 15% and preferably lower than 10%.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this specification, each of which is incorporated herein by reference in its entirety.

The foregoing description will be more fully understood with reference to the following Examples. Such Examples, are, however, exemplary of methods of practising the present invention and are not intended to limit the scope of the invention.

### EXAMPLES

### Example 1

### Antimicrobial Susceptibility Testing Report

### Introduction

Burkholderia pseudomallei is a gram-negative bacterium endemic to tropical and subtropical areas of the world [1]. It is the etiologic agent of melioidosis, a disease of varying clinical manifestation and severity [2-5]. B. pseudomallei is notorious for its resistance to a number of classes of antimicrobials, resulting in limited options for treatment [6-8]. Due to several major concerns, including the difficulty of treatment and severity of infection [9], B. pseudomallei is currently classified as a Tier 1 (previously Category B) Select Agent by the Centers for Disease Control and Prevention. In this study Applicants investigated the in vitro efficacy of antimicrobials against a collection of 20 B. pseudomallei strains from Thailand and Northern Australia.

Reagents, Materials & Strains
Lennox LB agar
   Fisher Scientific—Lennox LB
Mueller Hinton Broth
   BD—BBL Mueller Hinton II Broth (cation-adjusted)
Sterile Saline (0.85% NaCl)
   Fisher Scientific—Sodium Chloride
Alaxia Reagents
   Alaxia—Lactoferrin (Vial C)
   Alaxia—Control Solution
   Alaxia—Vial A
   Alaxia—Vial B
5,5′-Dithiobis(2-nitrobenzoic acid)
   Sigma-Aldrich
Tris Buffer
   Life Technologies
Tris Hydrochloride
   Life Technologies
Sodium Borohydride
   Sigma-Aldrich
Syringes
   BD—10 mL syringe
   B Braun—20 mL syringe
   B Braun—21G×1½″ needle
10 mL Polystyrene Sterile Serological Pipets
   Corning Life Sciences
Filter Units
   Millipore—Amicon Ultracel-30 Centrifugal Units
   Life Science Products—25 mm, 0.2 um Cellulose Nitrate
Pipette Tips
   USA Scientific—P1000 filtered tips
   USA Scientific—P200 filtered tips
   USA Scientific—P20 filtered tips
   Rainin—PIO filtered tips
Cuvettes & Lids
   Fisher Scientific—1.5 mL cuvette
   VWR—3 mL cuvette
   Carl Roth GmbH Co. KG—cuvette lids
Disposable Inoculating Loops 1/10 μL
   Fisher Scientific
Petri Dishes 100×13 mm
   Fisher Scientific
Reagent Reservoirs
   USA Scientific—12-well v-bottom
   VWR—25 mL polystyrene
TPP 96-well microtitre plates
   Sigma-Aldrich—MIC plates

TABLE 1
Burkholderia pseudomallei isolates used in this study.

| | Specimen | Year of | |
|---|---|---|---|
| Strain | Source | Isolation | Location |
| 1026b | blood | 1993 | Thailand |
| K96243 | clinical | 1996 | Thailand |
| 406e | wound | 1988 | Thailand |
| 1106a | pus | 1993 | Thailand |
| NCTC | clinical | 1996 | Thailand |
| 13392 | | | |
| 2613a | blood | 2001 | Thailand |
| 2614a | pus | 2001 | Thailand |
| 2617a | pus | 2001 | Thailand |
| 2618a | pus | 2001 | Thailand |
| 2625a | blood | 2001 | Thailand |
| HBPUB10303a | sputum | 2011 | Thailand |
| HBPUB10134a | sputum | 2010 | Thailand |
| MSHR 305 | brain | 1994 | Australia |
| MSHR 435 | skin | 1996 | Australia |
| MSHR 465a | blood | 1997 | Australia |
| MSHR 491 | environmental | 1997 | Australia |
| MSHR 668 | blood | 1995 | Australia |
| MSHR 5848 | sputum | 2011 | Australia |
| MSHR 5855 | sputum | 2011 | Australia |
| MSHR 5858 | sputum | 2011 | Australia |

Methods

Minimal inhibitory concentration (MIC) values of hypothiocyanite (OSCN⁻) and bovine lactoferrin (bLF) were determined individually and in combination following the protocol described in Alaxia Work Instruction following Clinical and Laboratory Standards Institute (CLSI) guidelines 2012. Briefly, 256 mg/mL bLF stocks were made fresh daily, diluted to the specified concentrations, and added to 96-well plates containing the appropriate volumes of cation-adjusted Mueller Hinton broth (MHB) and control solution.

OSCN⁻ was synthesized according to Alaxia proprietary method and quantified according to TNB method as described in Alaxia Work Instruction following CLSI guidelines, filter sterilized, and the specified volumes were promptly added to microtitre plates.

B. pseudomallei inocula of the strains listed in Table 1 were prepared by direct colony suspension. Colonies were suspended from LB agar plates in MHB and diluted in 0.85% sterile saline to the turbidity of a 0.5 McFarland standard (OD_{600 nm} of 0.09-0.10). Immediately following the addition of OSCN⁻ to microtitre plates, bacterial suspensions were diluted 1:10 in MHB and 10 μl of these inocula were added to the experimental and positive control wells. Microtitre plates were incubated at 37° C. for 20 h, and MIC values were read at the point of complete growth inhibition. MIC testing for each strain was performed in biological triplicate on three separate days.

Results

MICs were determined for bLF and OSCN⁻ individually and in combination. Typically the mode of replicate results is reported, however since the OSCN⁻ synthesis yielded varying concentrations this was not possible. The MIC results (Table 2) for each compound alone represent the range of 3 replicates, while all of the combination results are reported.

The overall precision of the OSCN⁻ MIC results showed minimal variability. The greatest variation in MIC for a single isolate was 13.1 μg/mL (or a 1.26 fold variation). All of the B. pseudomallei isolates were inhibited by less than 63.3 μg/mL of OSCN⁻ alone. Many of the isolates were inhibited by even the lowest tested concentration of OSCN⁻.

Similarly, the variation of bLF MICs was very low. All of the B. pseudomallei isolates were capable of growth at concentrations as high as 91.429 mg/mL, which was the highest concentration tested.

In determining the MIC values for the combination, Applicants noted that none of the isolates were capable of growth beyond the 31.7 μg/mL of OSCN⁻ in the presence of bLF, with the exception of one replicate of strain MSHR 435 (45.8 μg/mL). The combination of OSCN⁻ and bLF appears to have reduced the OSCN⁻ concentrations required for inhibition of growth. However, OSCN⁻ alone MICs were below detection for many of the strains complicating the ability to directly compare the results of these experimental conditions.

TABLE 2
MIC results for B. pseudomallei isolates
Minimal Inhibitory Concentration (μg/mL)

| | | | OSCN⁻/bLF | | |
|---|---|---|---|---|---|
| Strain | OSCN⁻ | bLF | 1 | 2 | 3 |
| 1026b | 46.6-51.5 | >91,429 | 25.8/7619 | 23.4/3810 | 23.3/472 |
| K96243 | ≦45.3 | >91,429 | 22.7/7619 | ≦28.6/122 | ≦30.7/122 |
| 406e | 47.5-59.9 | >91,429 | 23.8/15238 | 28.4/472 | 29.9/914 |
| 1106a | ≦47.5 | >91,429 | 23.8/15238 | ≦28.4/122 | ≦29.9/122 |
| NCTC13392 | 48.7-61.2 | >91,429 | 24.3/914 | 30.6/472 | 30.5/1905 |
| 2613a | 44.2-49.5 | >91,429 | 24.8/3810 | 24.2/914 | 22.1/1905 |
| 2314a | ≦24.6 | >91,429 | 24.1/244 | ≦24.6/122 | ≦23.8/122 |
| 2317a | ≦47.6 | >91,429 | 24.1/472 | ≦24.6/122 | 23.8/472 |
| 2618a | ≦49.6 | >91,429 | ≦23.3/122 | 24.8/472 | ≦22.6/122 |
| 2625a | ≦46.8 | >91,429 | 23.3/472 | ≦24.8/122 | ≦22.6/122 |
| HBPUB10303a | ≦59.7 | >91,429 | ≦24.2/122 | 29.9/742 | ≦28.7/122 |
| HBPUB10134a | ≦29.9 | >91,429 | 24.2/244 | ≦29.9/122 | ≦28.7/122 |
| MSHR 305 | 56.6-51.5 | >91,429 | 25.8/3810 | 23.4/3810 | 23.3/914 |
| MSHR 491 | 45.1-49.4 | >91,429 | 24.7/3810 | 22.9/3810 | 22.5/30476 |
| MSHR 435 | 45.1-49.4 | >91,429 | 24.7/30476 | 45.8/122 | 22.5/7619 |
| MSHSR 668 | ≦57.2 | >91,429 | 22.7/7619 | 28.6/472 | ≦30.7/122 |
| MSHR 465a | 44.2-49.5 | >91,429 | 24.8/1905 | 24.2/15238 | 22.1/7619 |
| MSHR 5848 | 50.2-63.3 | >91,429 | 25.1/472 | 27.8/1905 | 31.7/472 |
| MSHR 5855 | ≦55.6 | >91,429 | 25.1/3810 | 27.8/1905 | ≦31.6/122 |
| MSHR 5858 | ≦30.6 | >91,429 | ≦24.3/122 | ≦30.6/122 | 30.5/244 |

### SUMMARY

Overall OSCN⁻ was effective in vitro to inhibit the growth of 20 tested B. pseudomallei isolates. While B. pseudomallei was not directly inhibited by bLF alone, the combination of bLF with OSCN⁻ may have lowered the concentration of OSCN⁻ required to inhibit growth.

As we can see above, adding lactoferrin to OSCN⁻ reduce OSCN⁻ MIC level.

### Example 2

### Antimicrobial Susceptibility Testing Report

### Introduction

In this study Applicants investigated the in vitro efficacy of hypothiocyanite (OSCN⁻) and bovine lactoferrin (bLF) against two clinical B. pseudomallei strains.

Reagents, Materials & Strains

Same as example 1 and
Square Gridded Petri Dishes 100×100 mm
   Fisher Scientific

TABLE 3
Burkholderia pseudomallei isolates used in this study

| | Specimen | Year of | |
|---|---|---|---|
| Strain | Source | Isolation | Location |
| 1026b | blood | 1993 | Thailand |
| MSHR 305 | brain | 1994 | Australia |

Methods

Kill curve analysis was performed to examine the inhibition of B. pseudomallei by OSCN⁻ and bLF separately and in combination. Experiments were performed as described in Alaxia Work Instruction following CLSI guidelines 1999, with modifications as described below. Kill curves were performed in biological triplicate on three separate days.

B. pseudomallei inocula of the strains listed in Table 3 were prepared by direct colony suspension. Colonies were suspended from LB agar plates in cation-adjusted Mueller Hinton Broth (MHB) and diluted in 0.85% sterile saline to the turbidity of a 0.5 McFarland standard (OD600 nm of 0.09-0.10).

Cation-adjusted MHB was used as the broth media in the sample tubes. bLF was tested at a final concentration of 4 mg/mL, diluted from 80 mg/mL stocks made fresh daily. OSCN⁻ was synthesized according to Alaxia proprietary method and quantified according to TNB method as described in Alaxia Work Instruction following CLSI guidelines, filter sterilized, and added the appropriate sample tubes for a final concentration of 80 μg/mL.

Immediately following the addition of OSCN⁻ and corresponding volumes of control solution, 200 μl of bacterial suspensions were added to sample tubes, vortexed at full speed for 10 seconds, and 80 μl was removed from each tube as the T₀ₕ sample. A negative control tube containing 2.5 mL MHB and 2.5 mL control solution was included with each biological replicate. Sample tubes were incubated at 37° C. with constant shaking at 275 rpm.

Samples taken at each time point (T₀ₕ, T₂ₕ, T₄ₕ, T₆ₕ, and T₂₄ₕ), were diluted by serial 10-fold dilutions in MHB in a 96-well microtitre plate, and 10 μl aliquots from these dilutions were spotted in triplicate on LB agar. After 18-24 hours at 37° C., colonies were counted and colony forming units (CFU)/mL were calculated for each time point and plotted on a log 10 graph.

Results

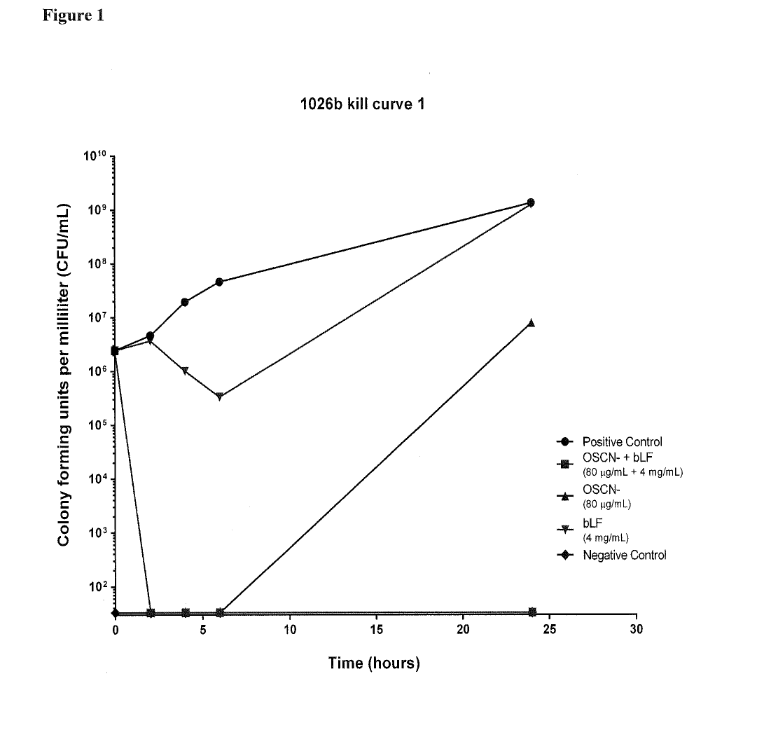

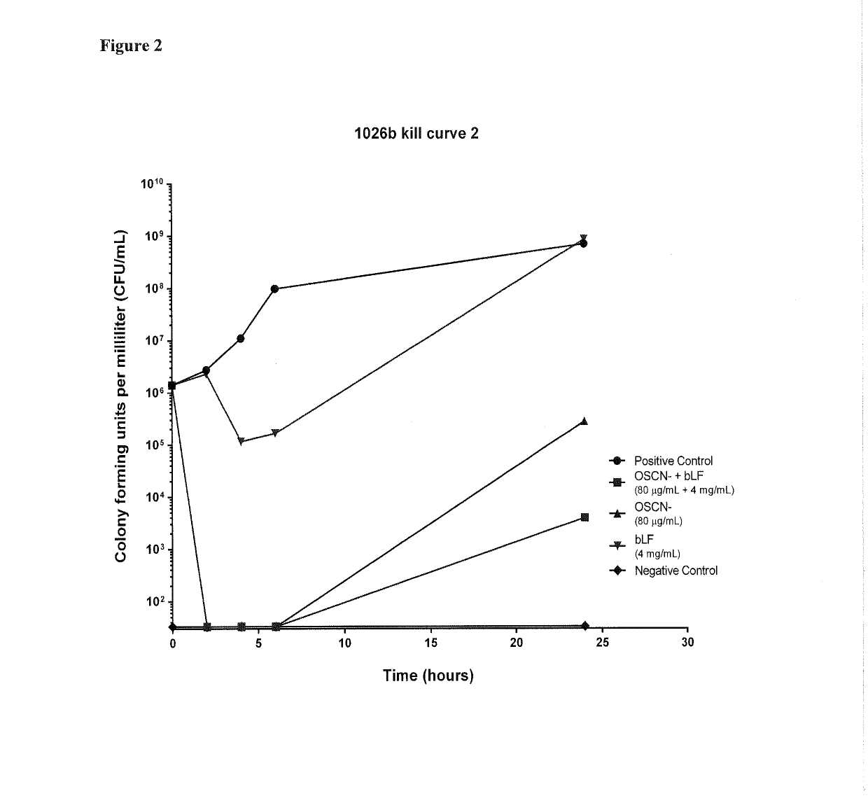

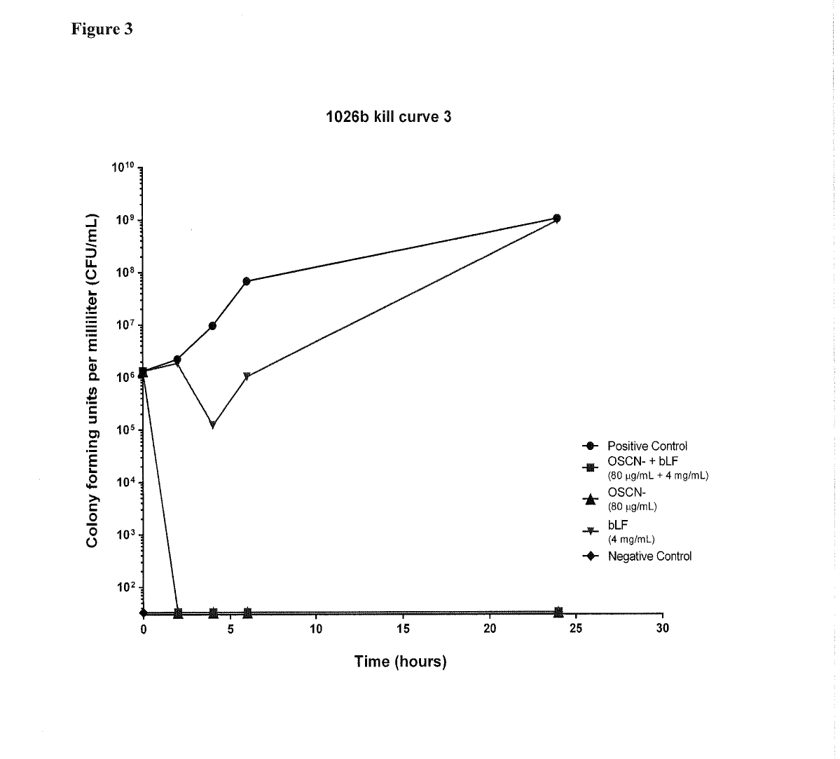

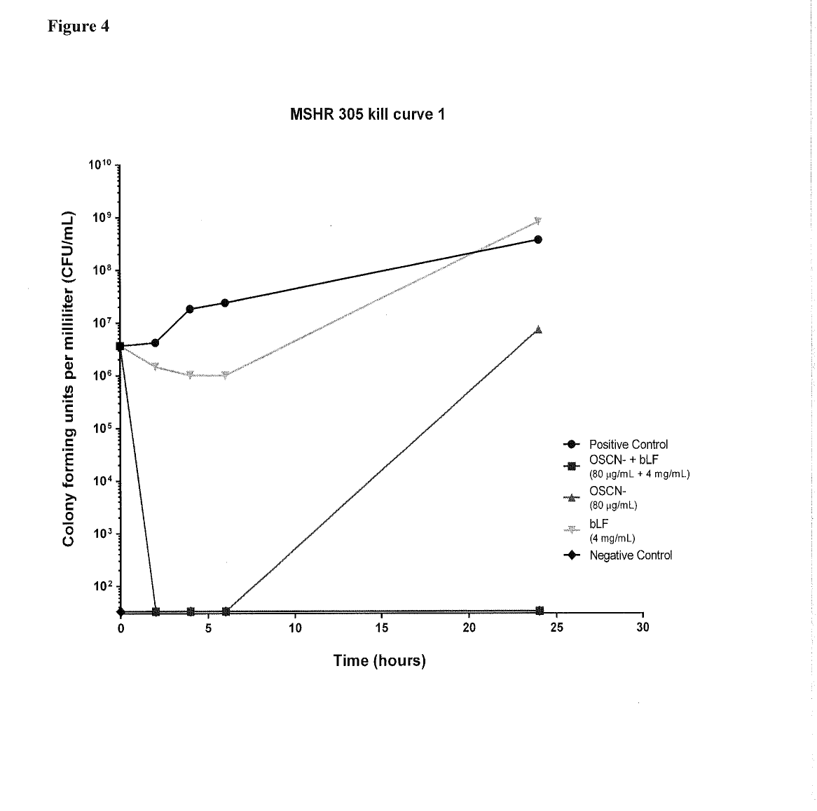

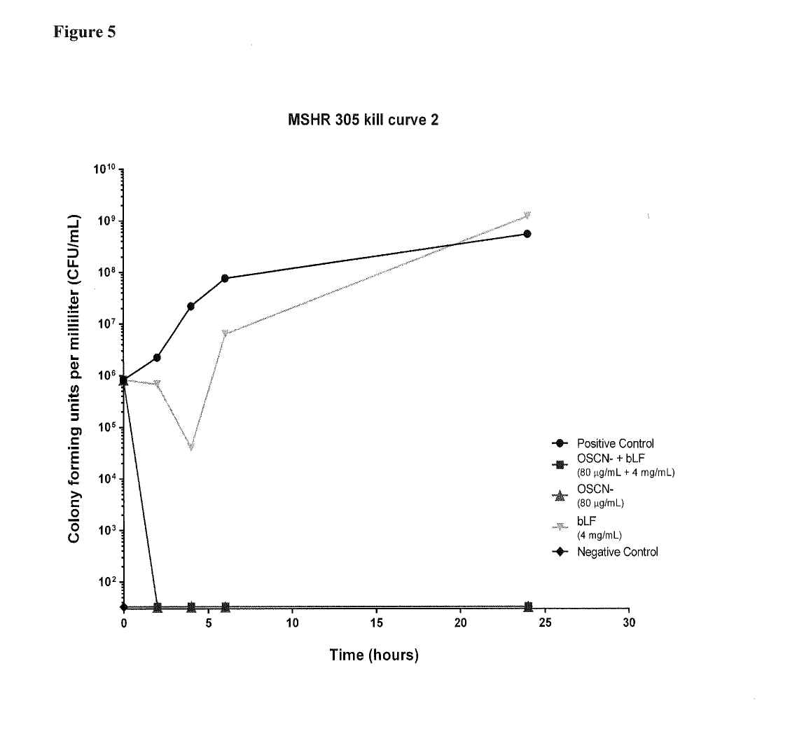

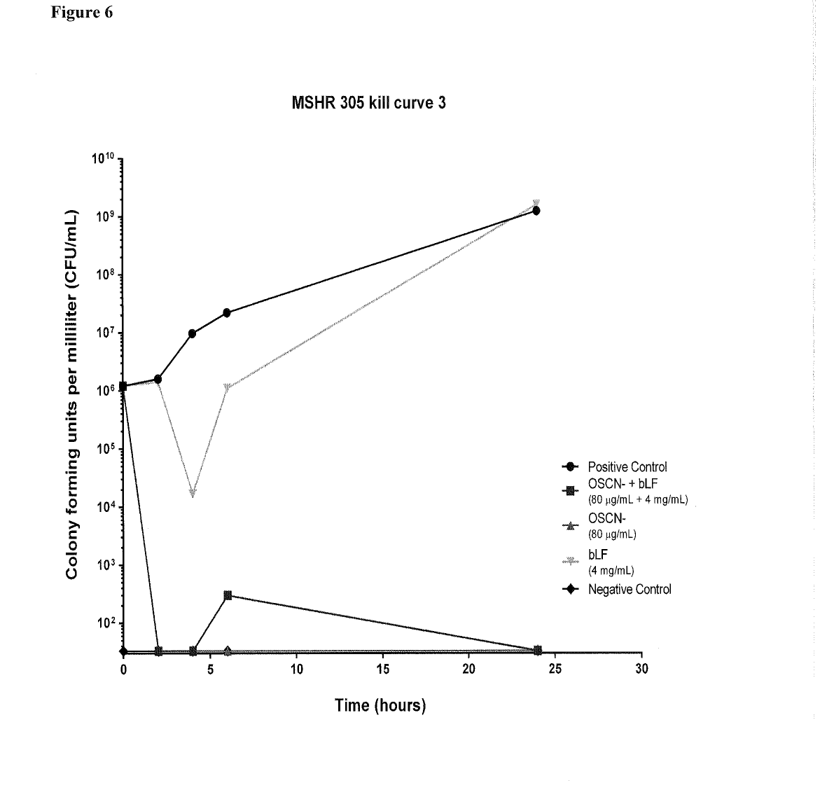

The kill curve assay was done in biological triplicate for each B. pseudomallei strain on three different days, and each dilution of the samples was plated in technical triplicate at each time point. A graphical representation of each kill curve is provided below, FIGS. 1-6.

The initial inoculum (T₀ₕ) was between 1.33×10⁶ and 2.43×10⁶ CFU/mL for 1026b and between 8.33×10⁵ and 3.67×10⁶ CFU/mL for MSHR 305. As expected, the bacterial counts in all of the positive control samples continued to increase throughout the time course, while the negative controls had no growth at any of the time points.

An initial 1 to 2 log₁₀ reduction in bacterial concentration was observed in the samples containing bLF alone for 4-6 hours post inoculation (T₂ₕ, T₄ₕ, T₆ₕ). However, after this initial phase, these samples grew to match or exceed the concentration of the positive control by T₂₄ₕ.

OSCN⁻ had a significant bactericidal effect. From T₂ₕ to T₆ₕ there were no detectable CFUs from either strain treated with OSCN⁻ or OSCN⁻+bLF, except for in one biological replicate of MSHR 305. This may have been erroneously introduced during mixing of the dilutions in the microtitre plate, considering also that there were no CFUs detected at T₂₄ₕ for this sample.

There was variability of results between biological replicates at T₂₄ₕ for the OSCN⁻ and OSCN⁻+bLF treated samples. During the initial kill curves (FIGS. 1 & 4) the OSCN⁻ alone samples for both strains had significant growth at T₂₄ₕ (though reduced by 1.5 to 2 log₁₀ as compared with the untreated controls). Up to 6 h contact time, microbial load impact reduction for OSCN⁻, OSCN⁻/bLF and bLF is reproducible.

During the second set of kill curves, both the 1026b samples treated with OSCN⁻ alone and OSCN⁻+bLF had growth at T₂₄ₕ, with approximately a 2.5 log₁₀ and 4.5 log₁₀ reductions compared with the untreated control, respectively (FIG. 2). However, no MSHR 305 growth was detected from either the OSCN⁻ or OSCN⁻+bLF samples at T₂₄ₕ (FIG. 5).

Finally, in the third set of kill curves (FIGS. 3 & 6) no growth was detected from either strain in the OSCN⁻ or OSCN⁻+bLF treated samples at T₂₄ₕ.

### SUMMARY

Overall, OSCN⁻ had a substantial bactericidal effect against the two B. pseudomallei strains tested, and bLF appears to have had a much more minor direct bactericidal effect, as evidenced by the initial decreases in bacterial concentrations in the samples treated with each compound individually.

While bacterial counts in the samples containing OSCN⁻ remained below the limit of detection (<33 CFU/mL) through 6 h post inoculation, some of these replicates did have significant bacterial growth by 24 h post inoculation. The final bacterial concentrations in the OSCN⁻ treated samples with growth at T₂₄ₕ were reduced by at least 1.5 log₁₀ as compared with the untreated controls. This suggests that OSCN⁻ was able to kill the majority of the bacteria in these samples, and that any few remaining cells that escaped the killing were able to grow to significant levels by 24 h post inoculation.

Despite the initial bactericidal effect seen with bLF alone, the bacteria that survived the initial inhibition were able to replicate to the same levels as those in the untreated controls by 24 h post inoculation. However, treatment with bLF and OSCN⁻ together provided increased inhibition over that of OSCN⁻ alone. With the exception of one replicate of one strain, the combination-treated samples were still culture-negative at 24 h post inoculation. The sample that did grow showed an approximately 2 log₁₀ reduction in bacteria compared with the corresponding OSCN⁻ only sample at T₂₄ₕ, and an approximately 5 log₁₀ reduction compared with the untreated control.

These results suggest that OSCN⁻ was able to kill the majority of the B. pseudomallei cells in these experiments, and the addition of bLF helped to kill some or all of the remaining organisms, thereby likely increasing the efficacy of treatment over either compound alone.

### Example 3

### Minimal Inhibitory Concentration Test Results for ALX-009 Against Select Agents

### Introduction

Bacillus anthracis, Burkholderia mallei, Francisella tularensis, and Yersinia pestis are the etiologic agents of anthrax, glanders, tularemia, and plague, respectively. Due to the severity of these infections and their potential use as biological weapons [9], these organisms are currently listed as Tier 1 Select Agents by the Centers for Disease Control and Prevention. In this study Applicants investigated the in vitro antimicrobial efficacy of hypothiocyanite (OSCN⁻) and bovine lactoferrin (bLF) individually and in combination against two strains of each of these organisms.

Reagents, Materials & Strains
Lennox LB agar
   Fisher Scientific—Lennox LB
Blood Agar (TSA w/ 5% Sheep Blood) plates
   Fisher Scientific—SBA plates
Chocolate Agar plates
   Teknova
Mueller Hinton Broth
   BD—BBL Mueller Hinton II Broth (cation-adjusted
IsoVitaleX
BD—BBL IsoVitaleX Enrichment
Sterile Saline (0.85% NaCl)
   Fisher Scientific—Sodium Chloride
Glycerol
   Fisher Scientific
Alaxia Reagents
   Alaxia—Lactoferrin (Vial C)
   Alaxia—Control Solution
   Alaxia—Vial A
   Alaxia—Vial B
5,5′-Dithiobis(2-nitrobenzoic acid)
   Sigma-Aldrich
Tris Buffer
   Life Technologies
Tris Hydrochloride
   Life Technologies
Sodium Borohydride
   Sigma-Aldrich
Syringes
   BD—10 mL syringe
   B Braun—20 mL syringe
Needles
   B Braun—21 G×1½″ needle
10 mL Polystyrene Sterile Serological Pipets
   Corning Life Sciences
Filter Units
   Millipore—Amicon Ultracel-30 Centrifugal Units
   Life Science Products—25 mm, 0.2 um Cellulose Nitrate
Pipette Tips
   USA Scientific—P1000 filtered tips
   USA Scientific—P200 filtered tips
   USA Scientific—P20 filtered tips
   Rainin—P10 filtered tips
Cuvettes & Lids
   Fisher Scientific—1.5 mL cuvette
   VWR—3 mL cuvette
   Carl Roth GmbH Co. KG
Disposable Inoculating Loops 1/10 μL
   Fisher Scientific
Petri Dishes 100×13 mm
   Fisher Scientific
Reagent Reservoirs
   USA Scientific—12-well v-bottom
   VWR—25 mL polystyrene
TPP 96-well microtitre plates
   Sigma-Aldrich—MIC plates

TABLE 4
Select Agent bacterial strains used in this study

| Organism | Strain |
|---|---|
| Bacillus anthracis | Ames |
| Bacillus anthracis | B286/76 |
| Burkholderia mallei | China 7 |
| Burkholderia mallei | A188 |
| Yersinia pestis | CO92 |
| Yersinia pestis | MG05 |

Methods

Minimal inhibitory concentration (MIC) values of OSCN⁻ and bLF were determined individually and in combination following the protocol described in Alaxia Work Instruction following CLSI guideline 2012. Briefly, 256 mg/mL bLF stocks were made fresh daily, diluted to specified concentrations, and added to 96-well plates containing the appropriate volumes of cation-adjusted Mueller Hinton broth (MHB) or modified-MHB (MMHB) and control solution. Filter-sterilized IsoVitaleX was added to MHB according to the manufacturer's instructions to generate MMHB. OSCN⁻ was synthesized according to Alaxia proprietary method and quantified according to TNB method as described in Alaxia Work Instruction following CLSI guideline 2012, filter sterilized, and the specified volumes were promptly added to microtitre plates.

Bacterial inocula were prepared by direct colony suspension, as described below. The bacterial suspensions were then diluted in 0.85% sterile saline to the turbidity of a 0.5 McFarland standard (OD600 nm of 0.09-0.10). Immediately following the addition of OSCN⁻ to microtitre plates, bacterial saline suspensions were diluted 1:10 in MHB or MMHB and 10 μl of these inocula were added to the experimental and positive control wells. Microtitre plates were incubated at 37° C. for 20-48 h as specified by the Clinical and Laboratory Standards Institute (CLSI) guidelines [10], and MIC values were read at the point of complete growth inhibition. MIC testing for each strain was performed in biological triplicate.

B. anthracis inocula: B. anthracis isolates were grown on either blood agar or Lennox LB plates at 37° C. overnight. Colonies were suspended in MHB and diluted to a 0.5 McFarland standard. Microtitre plates were incubated at 37° C. for 20 h.

B. mallei inocula: B. mallei isolates were grown on LB agar containing 4% glycerol at 37° C. for two days. Colonies were suspended in MHB and diluted to a 0.5 McFarland standard. Microtitre plates were incubated at 37° C. for 20 h.

Y. pestis inocula: Y. pestis isolates were grown on blood agar plates at 37° C. overnight. Colonies were suspended in MHB and diluted to a 0.5 McFarland standard. Microtitre plates were incubated at 37° C. for 24 h.

Results

MICs were determined for bLF and OSCN⁻ individually and in combination. Typically the mode of replicate results is reported, however since the OSCN⁻ synthesis yielded varying concentrations this was not possible. The MIC results for each compound alone represent the range of 3 replicates, while combination results of interest are reported individually.

B. anthracis: The MIC results for B. anthracis testing are shown below in Table 5. B. anthracis is unique from the other bacteria in this study as it is a Gram-positive organism, and is able to sporulate. MICs were determined on freshly grown culture (less than 23 h 10 m old) as well as on some older cultures (between 23 h 22 m and 46 h 53 m), which likely contained endospores. CLSI guidelines recommend incubating agar plates for 18 to 24 h for direct colony suspension [10].

B. anthracis strains tested from younger cultures were susceptible to low concentrations of OSCN⁻, with MICs at or below the detection limit. Conversely, even at very high concentrations of bLF there was no observed inhibition of growth.

MIC results for these strains did vary based on the age of the initial cultures. At the intermediate times there were significant differences in the OSCN⁻ and OSCN⁻/bLF combination for the B286/76 strain, and the addition of bLF appears to have enhanced the inhibition of OSCN⁻ at a given concentration. Whereas when testing from very old inocula we observed minimal inhibition of growth and no MIC could be determined for these strains.

TABLE 5
B. anthracis MIC results
Minimal Inhibitory Concentration (μg/mL)

| | Age of | | | | | |
|---|---|---|---|---|---|---|
| Strain | inoculum | OSCN⁻ | bLF | OSCN^{-/}bLF | | |
| Ames | Less than | ≦31.3 | >91,400 | ≦28.3/122 | ≦30.8/122 | ≦31.3/122 |
| B286/76 | 23 h 10 m | ≦31.3 | >91,400 | 23.9/472 | ≦28.3/122 | ≦31.3/122 |
| B286/76 | 23 h 22 m | 93.0- | >91,400 | 23.3/914 | 30.8/7619 | ND |
| | to | >153.8 | | 46.5/472 | 61.5/3810 | |
| | 23 h 49 m | | | 69.8/472 | 92.3/7619 | |
| | | | | | 123.1/1905 | |
| Ames | 46 h 53 m | >135.9 | >91,400 | >113.2/30476 | ND | ND |
| B286/76 | | ≦31.3 | >91,400 | >113.2/30476 | ND | ND |

ND, no data/not tested.

B. mallei: The MIC results for each compound alone and in combination are reported in Table 6. While the bLF generally did not inhibit B. mallei growth by itself, the OSCN⁻ was very effective against both strains tested. MIC values were at or below the limit of detection (≦25.9 μg/ml) for OSCN⁻ both with and without bLF.

TABLE 6
B. mallei MIC results
Minimal Inhibitory Concentration (μg/mL)

| Strain | OSCN⁻ | bLF | OSCN⁻/bLF |
|---|---|---|---|
| China 7 | ≦25.9 | ≧91,400 | ≦25.9/122 |
| A188 | ≦25.9 | ≧91,400 | ≦25.9/122 |

Y. pestis: The MIC results for each compound alone and in combination are reported in Table 7. While the bLF did not inhibit Y. pestis growth at any concentration by itself, the OSCN⁻ was very effective against both strains tested. MIC values were at or below the limit of detection (≦30.3 μg/ml) for OSCN⁻ both with and without bLF.

TABLE 7
Y. pestis MIC results
Minimal Inhibitory Concentration (μg/mL)

| Strain | OSCN⁻ | bLF | OSCN⁻/bLF |
|---|---|---|---|
| CO92 | ≦30.3 | >91,400 | ≦30.3/122 |
| MG05 | ≦30.3 | >91,400 | ≦30.3/122 |

### SUMMARY

Overall, OSCN appears to be very effective against the Gram-negative Select Agents tested, while the bLF by itself has no direct inhibitory effect. Due to the pre-determined quantities of compounds specified in the MIC protocol, the MIC values for OSCN⁻ were at or below the limit of detection for many of the organisms tested.

### REFERENCES

1. Currie, B. J., D. A. Dance, and A. C. Cheng, The global distribution of Burkholderia pseudomallei and melioidosis: an update. Trans R Soc Trop Med Hyg, 2008. 102 Suppl 1: p. S1-4.
2. Cheng, A. C. and B. J. Currie, Melioidosis: epidemiology, pathophysiology, and management. Clin Microbiol Rev, 2005. 18(2): p. 383-416.
3. Wiersinga, W. J., et al., Melioidosis: insights into the pathogenicity of Burkholderia pseudomallei. Nat Rev Microbiol, 2006. 4(4): p. 272-82.
4. Currie, B. J., L. Ward, and A. C. Cheng, The epidemiology and clinical spectrum of melioidosis: 540 cases from the 20 year Darwin prospective study. PLoS Negl Trop Dis, 2010. 4(11): p. e900.
5. Limmathurotsakul, D. and S. J. Peacock, Melioidosis: a clinical overview. Br Med Bull, 2011. 99: p. 125-39.
6. Wuthiekanun, V. and S. J. Peacock, Management of melioidosis. Expert Rev Anti Infect Ther, 2006. 4(3): p. 445-55.
7. Lipsitz, R., et al., Workshop on treatment of and postexposure prophylaxis for Burkholderia pseudomallei and B. mallei infection, 2010. Emerg Infect Dis, 2012. 18: p. e2.
8. Schweizer, H. P., Mechanisms of antibiotic resistance in Burkholderia pseudomallei: implications for treatment of melioidosis. Future Microbiol, 2012. 7(12): p. 1389-99.
9. Rotz, L. D., et al., Public health assessment of potential biological terrorism agents. Emerg Infect Dis, 2002. 8(2): p. 225-30.
10. CLSI, Performance Standards for Antimicrobial Susceptibility Testing; Twentieth Informational Supplement, in CSLI document M100-S202010, Clinical and Laboratory Standards Institute: Wayne, Pa.

1. A method for therapeutic or prophylactic treatment of melioidosis and/or associated diseases in a subject in need thereof, comprising administering to said subject an effective amount of a pharmaceutical composition comprising either:
a) one ion selected from the group of the hypothiocyanites (OSCN⁻) and/or hypohalites and/or
b) lactoferrin,
or a combination thereof.

2. The method of claim 1, wherein the pharmaceutical composition consists of a synergistic combination of at least one ion selected from the group of the hypothiocyanites (OSCN⁻) and/or hypohalites and lactoferrin.

3. The method of claim 1, wherein the associated diseases are bacterial infections selected from the group consisting of Burkholderia pseudomallei, Burkholderia mallei, bacillus anthracis, Yersinia pestis, and francisella tularensis infections.

4. The method of claim 1, wherein the ion is the hypothiocyanite ion.

5. The method of claim 1, wherein the ion is the hypoiodite ion (On.

6. The method of claim 1, wherein the lactoferrin is a lactoferrin having a purity higher than 97%, essentially free from endotoxin, lipopolysaccharide and angiogenin and with an iron saturation level lower than 15%.

7. The method according to claim 1, wherein pharmaceutical composition further comprises an antibiotic or a therapeutic agent.

8. The method according to claim 7, wherein said antibiotic is commonly used for the prevention or treatment of bacterial infections selected from the group consisting of Burkholderia pseudomallei, Burkholderia mallei, bacillus anthracis, Yersinia pestis, and francisella tularensis infections.

9. The method according to claim 7, wherein said antibiotic is selected from the group consisting of piperacillin, ceftazidime, temocillin, carbapenem, imipenem, meropenem, rifampicin, tobramycin, ciprofloxacin, monosulfactam, amoxicillin, carbenicillin, Doxycycline, penicillin, Trimethoprim-sulfamethoxazole, monobactam, Streptomycin, Fosfomycin, Ethionamide, Isoniazid, Pyrazinamide, Rifampicin, Rifabutin, Rifapentine, Arsphenamine, Chloramphenicol, Fusidic acid, Metronidazole, Mupirocin, Platensimycin, Quinupristin/Dalfopristin, Thiamphenicol, Tigecycline, Tinidazole, Trimethoprim or their mixtures.

10. A method for therapeutic or prophylactic treatment of melioidosis and/or associated diseases, wherein for local treatment of the pulmonary epithelium the method comprises the administration of a therapeutically active quantity of at least one ion selected from the group of the hypothiocyanites and/or hypohalites and/or lactoferrin or a combination thereof.

11. The method of claim 10, wherein the associated diseases are bacterial infections selected from the group consisting of Burkholderia pseudomallei, Burkholderia mallei, bacillus anthracis, Yersinia pestis, and francisella tularensis infections.

12. The method of claim 10, wherein the method further comprises the administration of an antibiotic.

13. The method according to claim 12, wherein said antibiotic is commonly used for the prevention or treatment of bacterial infections selected from the group consisting of Burkholderia pseudomallei, Burkholderia mallei, bacillus anthracis, Yersinia pestis, and francisella tularensis infections.

14. The method according to claim 12, wherein said antibiotic is selected from the group consisting of piperacillin, ceftazidime, temocillin, carbapenem, imipenem, meropenem, rifampicin, tobramycin, ciprofloxacin, monosulfactam, amoxicillin, carbenicillin, Doxycycline, penicillin, Trimethoprim-sulfamethoxazole, monobactam, Streptomycin, Fosfomycin, Ethionamide, Isoniazid, Pyrazinamide, Rifampicin, Rifabutin, Rifapentine, Arsphenamine, Chloramphenicol, Fusidic acid, Metronidazole, Mupirocin, Platensimycin, Quinupristin/Dalfopristin, Thiamphenicol, Tigecycline, Tinidazole, Trimethoprim or their mixtures.
